(19) United States
(12) Patent Application Publication
(10) Pub. No.: US 2006/0165821 A1
(43) Pub. Date: Jul. 27, 2006

(54) Composition comprising scutellaria baicalensis and their uses thereof

(76) Inventors: Yong Zhang, Jamaica, NY (US); Fei Ye, Beijing (CN); Fan Li, Beijing (CN)

Correspondence Address: STROOCK & STROOCK & LAVAN LLP, 180 MAIDEN LANE, NEW YORK, NY, 10038, US

(21) Appl. No.: 10/540,817

(22) PCT Filed: Dec. 26, 2003
(86) PCT No.: PCT/CN03/01134

§ 371 Date: Dec. 16, 2005

(30) Foreign Application Priority Data
Dec. 27, 2002 (CN) 021590214

(51) Int. Cl. A61K 36/539; A61K 31/737; A61K 31/7008

(52) U.S. Cl. 424/741; 514/62; 514/54

(57) ABSTRACT

This invention described various formula composite of Scutellaria baicalensis for various diseases and conditions. The formula takes the advantage of our recent discovery of Scutellaria baicalensis can inhibit cyclooxygenase-2 (COX-2) activity and expression, resulting in decrease of prostaglandin synthesis. COX-2 is involved in many biological processes, such as inflammation, pain, carcinogenesis, etc. Combination of Scutellaria baicalensis with lycopene can be used for cancer prevention, especially smoking induced. Combination of Scutellaria baicalensis with glucosamine and chondroitin can be used for arthritis. Finally, this invention disclosed various method to prepare this formula and composition.

### BACKGROUND OF THE INVENTION

1. Cancer Treatment of Scutellaria baicalensis and Other Herbs

Cancer is the third major cause of mortality, accounting for more than 7 million deaths per year world wide. Prostate cancer is the most common malignancy and the second leading cause of cancer deaths in man in the united state. The incidence of squamous cell cancer of head and neck is approximately 70,000 cases per year in United State, accounting for approximately 16,000 deaths in U.S per year. Colorectal is the second leading cause of cancer deaths in the United State. So, the three cancers are among the most morbid of human cancers in U.S. However, the standard treatments of radiation, chemotherapy and surgery for the three cancers have failed to improve patient outcome for the last decades, the main reason for failure methods is serious side effects. Therefore, improvements in the quality of life and survival in the patients with the cancers will require the development of better therapeutic agents. For this reason, cancer patients are turning to complementary and alternative medicine for therapeutic and palliative care. Recent survey has shown that up to 60% cancer patients use complementary and alternative therapies including herbal remedies. Herbs have been used as medicines in China and East Asia for several thousand years. But many herbs were used based on anecodotal experience, their efficacy and safety has not been scientifically evaluated.

Scutellaria baicalensis, also known as Chinese skullcap or Huang Qin, is a widely used herb in Traditional Chinese Medicine with anticancer, antiviral, antibacterial and anti-inflammatory properties (Razina, T et al. Vopr Onkologii, 35: 331-335, 1989). Inhibitory effects of flavonoids from Scutellaria baicalensis on Epstein-barr virus activation and their anti-tumor-promoting activities (Chem Pharm Bull, 40: 531-533, 1992.). Historically, Scutellaria baicalensis has been used to treat respiratory tract infection, diarrhea, jaundice, hepatitis, and cancer. Recent investigations have shown that Scutellaria baicalensis alone, or in combination with other herbs, can inhibit cancer cell growth or induce apoptosis in breast, hepatocellular, pancreatic, prostatic, and urothelial carcinoma cell lines (So, F. et al Cancer Lett, 112: 127-133, 1997. Yano, H. et al. Cancer Res, 54: 448-454, 1994). We also demonstrated that Scutellaria baicalensis extract exhibits a strong anticancer activity in vitro and in vivo, especially on head and neck squamous cell carcinoma that is usually resistant to chemotherapy (Zhang et al. Cancer Res 63:4037-4043, 2003). Inhibition of PGE₂ synthesis via suppression of COX-2 expression and activity and suppression of cancer cell proliferation via cell cycle arrest may be responsible for its anticancer activity.

2. Chemoprevention of Scutellaria baicalensis and Lycopene

Head and neck cancer, the fifth most common cancer worldwide, is an important contributor to cancer morbidity and mortality in the US (Bhuvaneswari V, et al. Fitoterapia, 2001, 72(8): 865-74). Despite advances in radiotherapy and chemotherapy, the survival of patients with head and neck squamous cell carcinoma (HNSCC) has not improved significantly over the past three decades. The main reasons for treatment failure are the development of second primary tumors in individuals with early-stage disease and local recurrences and metastases in patients with advanced HNSCC (Day G L et al. Cancer 1992, 70:14-19). Chemoprevention, the administration of natural or synthetic substances to prevent, delay or reverse carcinogenesis, represents a therapeutic strategy that may prove useful in individuals at increased risk for HNSCC or second primary tumors. To develop rational therapeutic stragies, it is important to identify molecular targets that are linked to the pathogenesis of HNSCC. Currently, cyclooxygenase-2 (COX-2), an inducible enzyme that catalyzes the synthesis of prostaglandins (PG), is being intensively evaluated as a potential molecular target for chemopreventive agents (Dannenberg A J et al. Lancet Oncol 2001, 2: 544-551). Furthermore, COX-2 has been found to be much higher expressed in squamous cell carcinoma than normal squamous cells. (Muller-Decker K, et al. Proc Natl Acad Sci USA, 2002. 99(19): p. 12483-8). We have recently demonstrated that Scutellaria baicalensis significantly reduced PGE2 synthesis via inhibition of COX-2 activity as well as COX-2 protein expression in SCC-25 cells in a dose-dependent manner. Thus, Scutellaria baicalensis maybe useful in preventing HNSCC.

The potential of single chemopreventives is limited by its potency. Simultaneous or sequential administration of multiple agents can increase efficacy. For example, differences in the chemopreventive mechanisms among the agents can provide additive or synergistic efficacy. Lycopene, a natural antioxidant found predominantly on tomato products, is attracting attention as a cancer prevention agent. Data suggest that the chemoprevention of lycopene is thought to be primarily responsible for its anti-oxidative property. Therefore, Scutellaria baicalensis in combination with lycopene may have potent synergistic effect in chemoprevention of HNSCC, based on different mechanisms among the both herbs. Other herbs or nutritional supplements which hav different biological activity can be incorporated with Scutellaria baicalensis for various cancer prevention.

3. Arthritis Treatment of Scutellaria baicalensis and Glucosamine/Chondroitin Sulfate

Arthritis is a widespread, potentially disabling disease, which is prevalent and clinical significant health care problems in the United States today, affecting 15% of the population (Brennan M R, et al. Annals of Internal medicine, 2003, 138, p 795). The most typical types of arthritis are osteoarthritis and rheumatoid arthritis, and the former one is more prevalent than the latter one. According to the American Arthritis Foundation, 45 million Americans of various ages or 80%-95% of those senior over 65 years old suffer from osteoarthritis that most commonly affects weight-bearing joints (e.g. hips, knees). It is important to have this in the mind how to most effectively treat osteoarthritis, especially among older people, because the risk of being affected by osteoarthritis increases with age.

For more than 30 years, non-steroidal anti-inflammatory drugs (NSAIDs) have been used as standards in the treatment of osteoarthritis (OA). However, the use of these drugs is limited primarily by their toxicity. Nonselective NSAIDs (i.e. those that inhibit both cyclooxygenase [COX]-1 and COX-2) are associated with an increased risk for serious upper gastrointestinal (GI) complications, including perforation, symptomatic ulcers and bleeding; nephrotoxicity, acute renal insufficiency and congestive heart failure. The recent introduction in the US of oral COX-2-specific inhibitors such as celecoxib (in 1999), rofecoxib (in 1999), and valdecoxib (in 2001), which selectively inhibit COX-2, are increasingly being used in place of nonselective NSAIDs. This is because they are just as effective as NSAIDs in relieving arthritic pain and yet less gastrotoxic. However, many studies showed that the COX-2 inhibitors may increase the risk of cardiovascular and renal diseases, compared with NSAIDs.

The use of combination glucosamine sulfate (GS) and chondroitin sulfate (CS) is becoming commonplace as one of complementary therapy for treatment of OA. OA is a progressive disease caused by the cartilage destruction and a progressive degeneration of cartilage glycosaminoglycans with subsequent formation of bone spurs. Whereas GS is thought to promote the formation and repair of cartilage, CS is believed to promote water retention and elasticity in cartilage and inhibit enzymes that break down cartilage. The combinatory use of GS and CS can provide substrates for proteoglycans synthesis. However, successful treatment of OA must effectively decrease and control pain and maintain or improve joint function. GS and CS have very little direct anti-inflammatory effect and no demonstrated ability to directly act as an analgesic or pain relieving agent. Instead, GS and CS appear to only promote the regeneration of the joint matrix by stimulating production of proteoglycans.

Scutellaria baicalensis, on the other hand, has a strong anti-inflammatory effect by inhibition of COX-2 pathway. It can reduce inflammation which destructs joint cartilage as well as pain. Therefore, we formulated an anti-arthritic nutritional supplement, containing SB, GS and CS. Our study demonstrated that this formula is much more effective and stronger in the treatment of OA than any mono-drug in animal study and in a limited clinical study.

The preferred anti-arthritic formula is consisted of 20-40% Scutellaria baicalensis extract, 30-60% Glucosamine sulfate and 10-20% Chondroitin sulfate. The preferred formulation is capsule or tablet. All these components can be purchased in US. The Scutellaria baicalensis extract is the root portion of the plant Scutellaria baicalensis Georgi which is preferably prepared by boiling the dried raw herb root in water or soaking in absolute ethanol followed by spray-drying to obtain a 12-fold concentration as compared to raw herb. The preferred adult dose of the formula is 1.5-2 grams per times, 2 times daily.

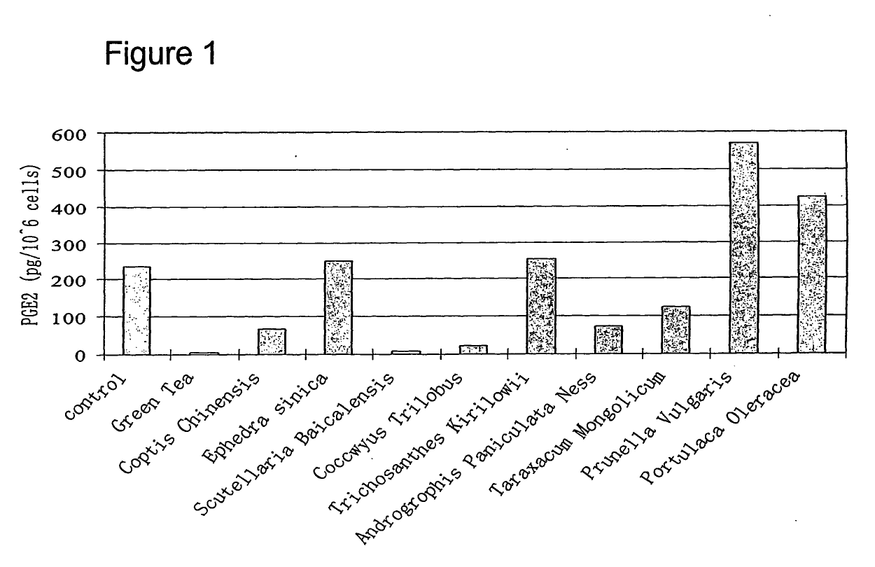

Other herbal extracts have been found that can inhibit PGE2 synthesis may also be used to substitute for Scutellaria baicalensis. These herbs include Green tea, Coptis chinensis, Coccwyus trilobus and Androgrophis Paniculata Ness (FIG. 1).

### BRIEF SUMMARY OF THE INVENTION

Scutellaria baicalensis extract exhibits a strong anticancer activity in vitro and in vivo, especially on head and neck squamous cell carcinoma. Inhibition of PGE₂ synthesis via suppression of COX-2 expression and activity and suppression of cancer cell proliferation via cell cycle arrest may be responsible for its anticancer activity. In addition, no side effect or toxicity was observed by Scutellaria baicalensis extract.

Scutellaria baicalensis maybe useful in preventing HNSCC, and Scutellaria baicalensis, in combination with other herbal and nutritional supplements including but not limited lycopene, may have potent synergistic effect in chemoprevention of HNSCC, based on different mechanisms among the herbs.

Scutellaria baicalensis in combination with other nutrition and herbal and nutritional supplements, including but not limited glucosamine and chondroitin, has a strong anti-inflammatory effect through the inhibition of COX-2 pathway. Furthermore, it can lessen the cartilage degeneration of the joints. Therefore, it can be effectively used in the treatment of OA which inflict millions of Americans. Its anti-arthritic effect and pain relieving effect had been confirmed in animal study and in a limited clinical study.

### DETAILED DESCRIPTION OF FIGURES

FIG. 1. Inhibition of PGE₂ synthesis in SCC-25 cells by various herbal extracts.

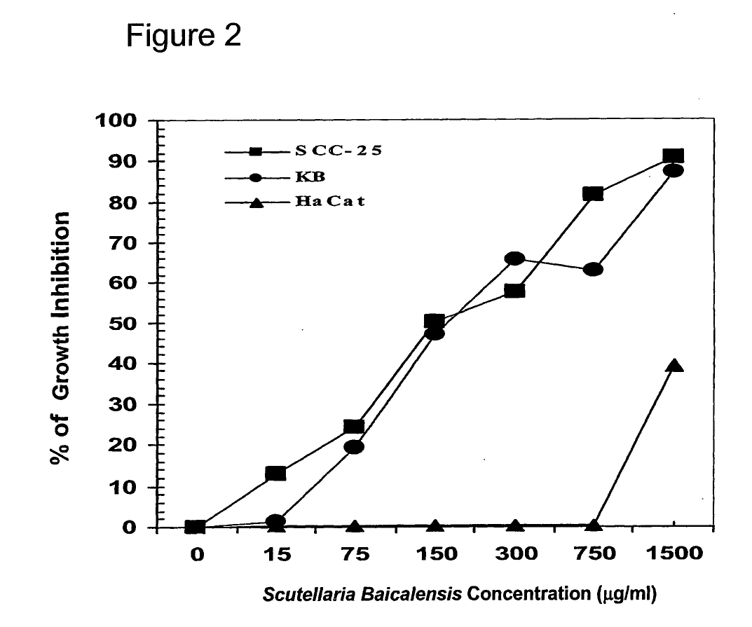

FIG. 2. Inhibition of HNSCC and HaCat cell growth by Scutellaria baicalensis.

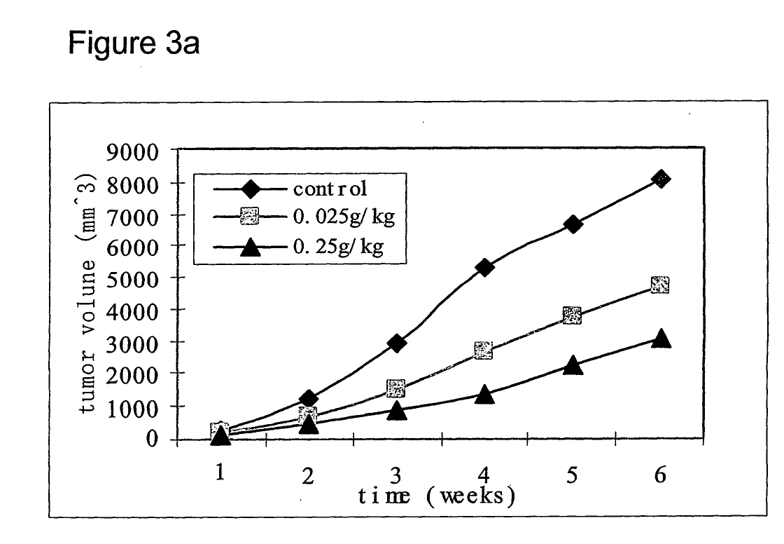

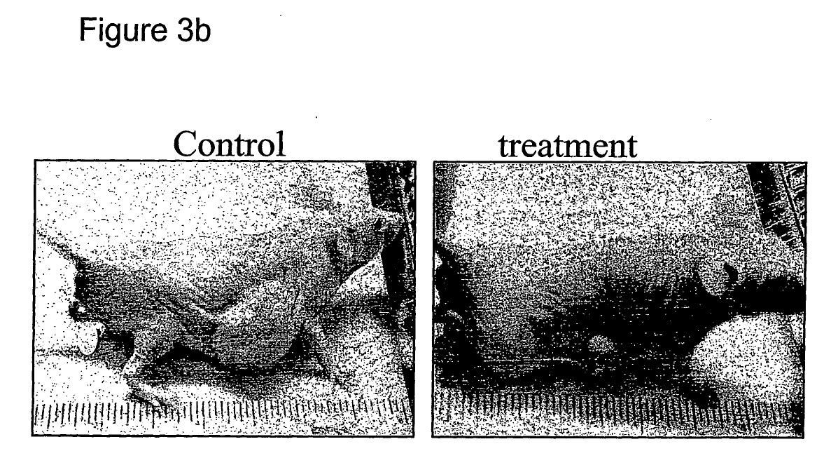

FIG. 3: Tumor growth inhibition in nude mice bearing KB cells. Panel A: tumor growth inhibition. Panel B: comparison of tumor size in control and treated mouse

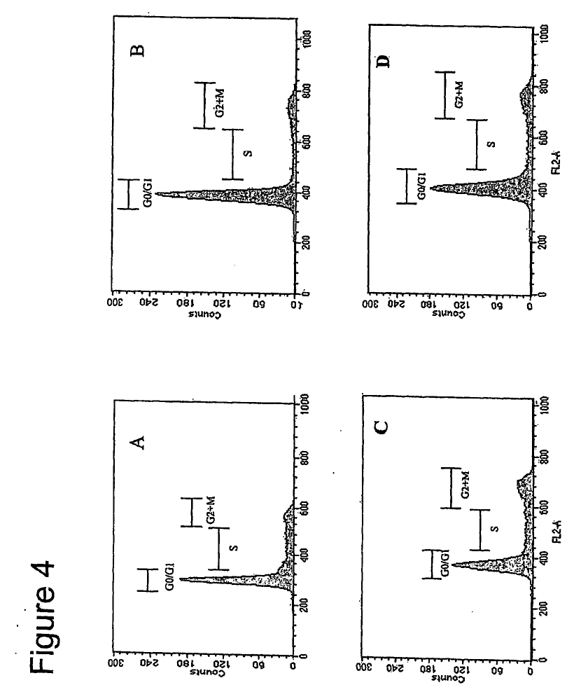

FIG. 4. Cell cycle analysis by flow cytometry: The SCC-25 (A and B) and KB (C and D) cells were treated with 750 ug/ml of Scutellaria baicalensis for 72 hours (B and D) and cells were harvested and subjected to flow cytometry analysis. The histograms show that there was a significant increase of G0/G1 population with concomitant decrease of S population after treatment with Scutellaria baicalensis (B and D).

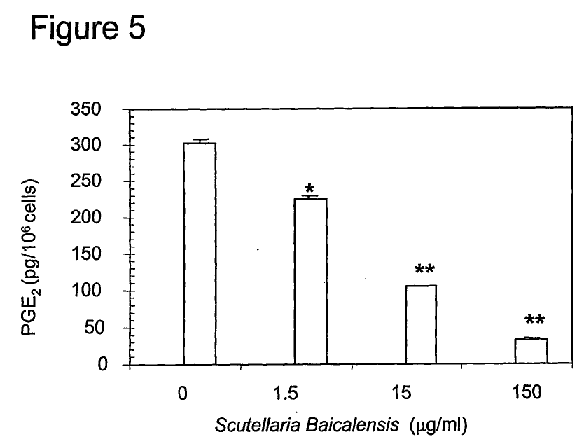

FIG. 5: Inhibition of PGE₂ synthesis in SCC-25 cells by Scutellaria baicalensis.

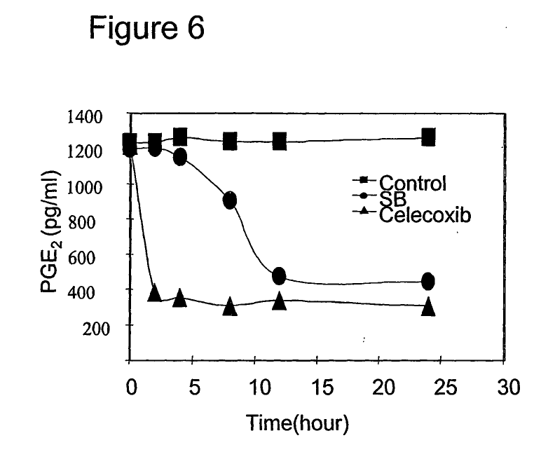

FIG. 6: Inhibition of COX-2 activity in SCC-25 cells by Scutellaria baicalensis (150 ug/ml) and Celecoxib (25 uM). Cells were incubated with either agent for various periods of time and the medium was removed. Fresh medium containing 100 uM arachidonic acid was added and incubated for additional 30 min. The level of PGE₂ in the medium was measured by EIA assay.

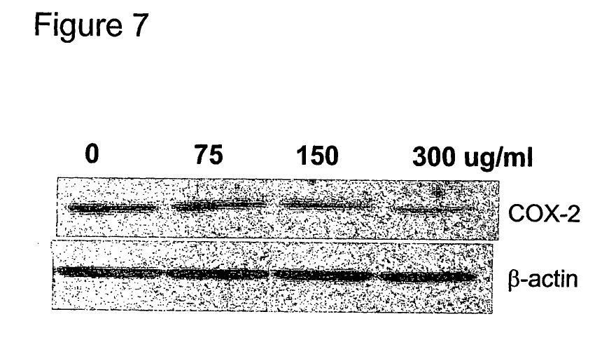

FIG. 7. Inhibition of COX-2 expression by Scutellaria baicalensis. SCC-25 cells were treated with Scutellaria baicalensis for 72 hours and proteins were fractionated through a SDS-PAGE and detected by Western blot.

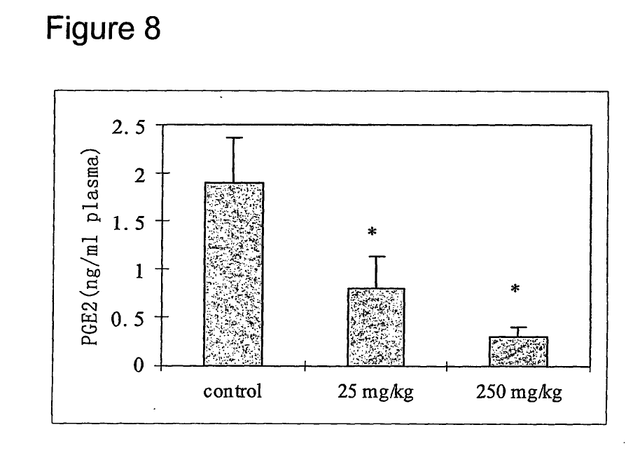

FIG. 8: inhibition of PGE₂ synthesis in the plasma of nude mice bearing squamous cell carcinoma by Scutellaria baicalensis (SB)

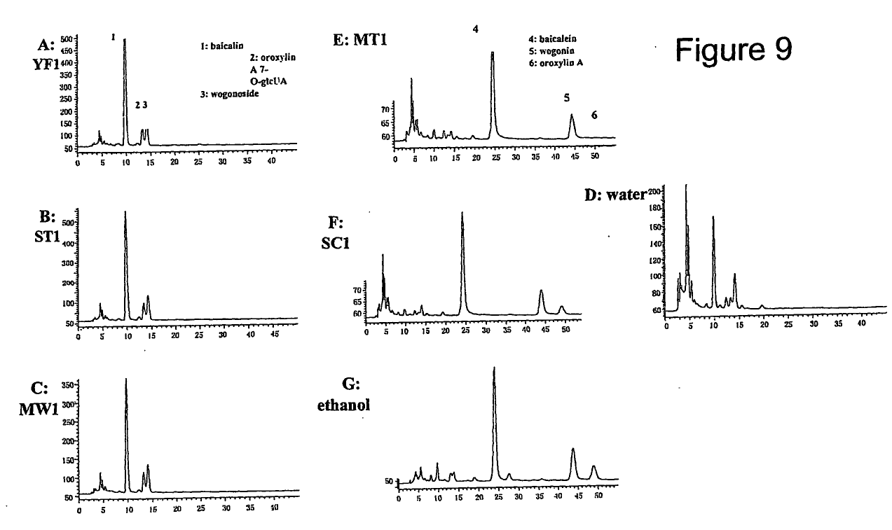

FIG. 9: HPLC profile of Scutellaria baicalensis extracts. A-C: commercial extracts from YF1, ST1, and MW1. E and F: commercial extracts from MT1 and SC1. D and G: extracts from our laboratory via water or ethanol

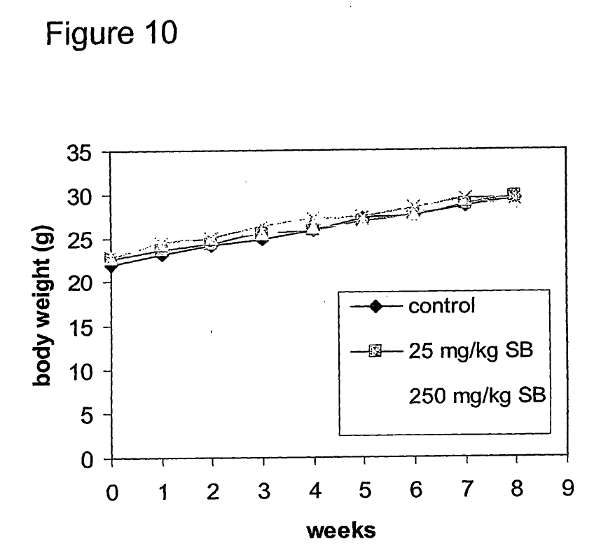

FIG. 10. The body weight of nude mice treated with Scutellaria baicalensi for 8 weeks

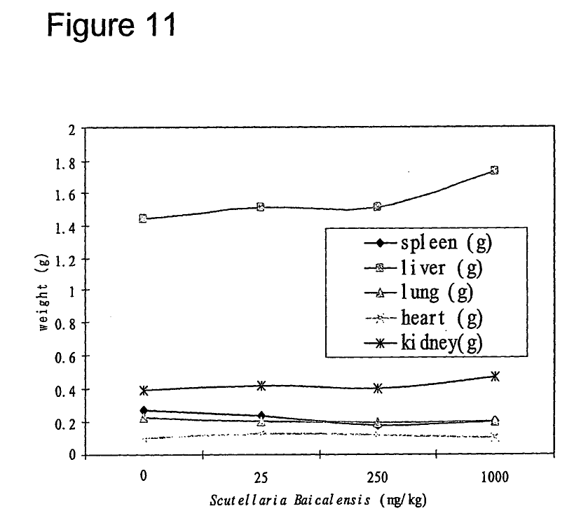

FIG. 11. The five vital organ weight of nude mice treated with Scutellaria bcaicalensis for 8 weeks

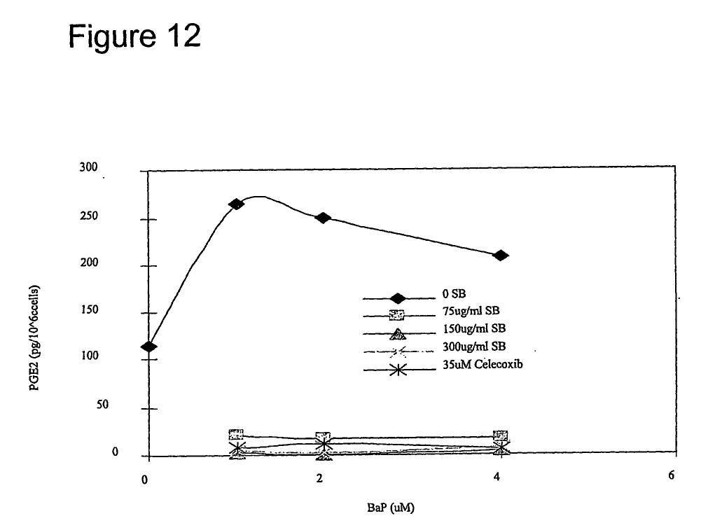

FIG. 12: Induction of PGE₂ synthesis by BaP and suppression of BaP-induced PGE₂ synthesis by Scutellaria baicalensis (SB) and celecoxib.

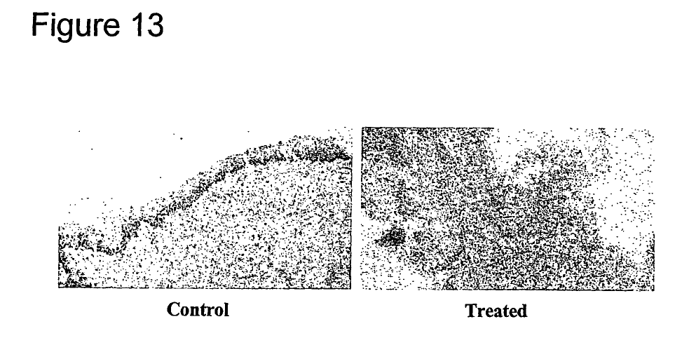

FIG. 13: Induction of hyperkeratosis by BaP in nude mice.

### DETAILED DESCRIPTION OF THE INVENTION

This invention provides a composition comprising an effective amount of Scutellaria baicalensis or an extract thereof. As defined herein the Scutellaria baicalensis extract can be obtained by routine procedure. In an embodiment, it is an aqueous extract.

This invention provides a composition of the above composition wherein the composition is capable of treating and/or preventing of cancer as used herein. The cancer may include prostate, breast, colon, liver, lung, pancreatic, head & neck, and liver cancers.

This invention provides a composition comprising Scutellaria baicalensis, Glucosamine and Chondroitin effective in preventing or treating arthritis. This arthritis includes but is not limited to rheumatoid arthritis, lupus-associated arthritis, osteoarthritis, and neuropathic arthropathy.

This invention provides the composition of the above described composition, wherein the arthritis is a rheumatoid arthritis and osteoarthritis.

This invention provides the composition of the above described wherein the 20-40% of Scutellaria baicalensis, 30-60% of Glucosamine and 10-20% of Chondroitin.

This invention provides the above composition wherein the proportion of Scutellaria baicalensis is about 34%, Glucosamine about 52% and Chondroitin about 14%.

This invention provides the method of treating a subject comprising administering to the subject an effective amount of the composition of the above described composition. In an embodiment, the subject is a human. In another embodiment, the subject is a mammal. In a further embodiment, the subject is a human-redundant.

This invention provides the method of treating a subject with arthritis comprising administering to the subject an effective amount above composition.

This invention provides a method for preparing the above composition.

This invention provides a formulation comprising the above composition.

This invention provides a container comprising the above composition.

This invention provides a method for quality control or batch variation of the above composition comprising the steps of:

(a) Physical characterization of said composition and

(b) Biological array of said composition.

This invention provides the above method wherein step (a) comprising high pressure chromatography.

This invention may be further understood by the following examples and claims. Nevertheless, in no circumstance, the examples should be limiting to this invention.

### Example 1

### Cancer Treatment of Scutellaria baicalensis Extract and Other Herbs

### Example 1.1

### Growth Inhibition of Scutellaria Baicalensis on Head and Neck Squamous Carcinoma (HNSCC) Cell Lines

The percentage of growth inhibition was determined by employing an MTT (3,4,5-dimethylthiazol-2,5-diphenyltetrazolium bromide; Sigma) assay to measure viable cells. Scutellaria baicalensis displayed a dose-dependent (FIG. 2) and time-dependent (not shown) inhibition on the growth of both HNSCC cell lines including KB and SCC-25 cell). Both cells exhibited equal sensitivity to Scutellaria baicalensis and 50% growth inhibition concentrations (IC₅₀) of both cell lines were determined to be 150 μg/ml (Zhang et al. Cancer Res 63:4037-4043, 2003). The maximal inhibition of cell growth (>90%) was achieved at 1500 μg/ml. On the other hand, Scutellaria baicalensis did not inhibit the growth of HaCaT cell, a nontumorigenic oral squamous cell line, even at a concentration of 750 μg/ml (FIG. 2), indicating that Scutellaria baicalensis selectively inhibited cancer cell growth.

### Example 1.2

### Inhibition of Tumor Growth in HNSCC-Xenografted Nude Mice

Nude mice inoculated subcutaneously with 3×10⁶ KB cells were treated daily by gavage for 8 weeks with Scutellaria baicalensis extract dissolved in water (Zhang et al. Cancer Res 63:4037-4043, 2003). Our results show that tumor volumes significantly decreased in the treated group in dose-dependant manner as compared with control group (FIG. 3). These results indicate that Scutellaria baicalensis not only inhibits cancer cell growth in vitro but also inhibits cancer growth in animals.

### Example 1.3

### Cell Cycle Arrest on HNSCC Cells by Scutellaria baicalensis

We further analyzed the effect of these agents on cell cycles (Table 1). Our results show that Scutellaria baicalensis caused a significant G₀/G₁ phase arrest with concurrent decrease of S phase in both SCC-25 and KB cells, compared to the control cells (FIG. 4) (Zhang, D., et al., Cancer Res, 2003, 63 (14): p 4037-43). In addition, Scutellaria baicalensis exerted no effect on the cell cycle distribution of HaCaT cells up to the concentration of 750 μg/ml (data not shown).

TABLE 1
Cell cycle distribution of SCC-25 and KB cells
after treating with Scutellaria baicalensis

| | SCC-25 | | | KB | | |
|---|---|---|---|---|---|---|
| | G₀/G₁ | S | G₂/M | G₀/G₁ | S | G₂/M |
| | (%) | (%) | (%) | (%) | (%) | (%) |
| Control | 60.43 | 24.37 | 15.2 | 59.85 | 17.8 | 22.35 |
| Scutellaria | | | | | | |
| baicalensis | | | | | | |
| 150 ug/ml | 66.61 | 16.13 | 17.26 | 67.56 | 13.22 | 19.22 |
| 300 ug/ml | 67.63 | 17.2 | 15.17 | 70.62 | 10.49 | 18.89 |
| 750 ug/ml | 71.53 | 14.45 | 14.02 | 76.13 | 10.48 | 13.39 |

### Example 2

### Anti-Arthritic Effect of Scutellaria baicalensis

### Example 2.1

### Inhibition of PGE2 Synthesis and Suppression of COX-2 Activity and Expression by Scutellaria baicalensis

Since Scutellaria baicalensis has been historically used to treat inflammatory disorders, we hypothesized that its anti-arthritic activity may derive from inhibition of cyclooxygenase-2 (COX-2) pathway. COX-2 has been found to be highly expressed in SCC-25 cells, a squamous cell carcinoma cell line (Muller-Decker K, et al. Proc Natl Acad Sci USA, 2002. 99(19): p. 12483-8). Therefore, it was used to test the effect of Scutellaria baicalensis on the prostaglandin E₂ (PGE₂) synthesis by EIA assay (Zhang, D., et al., Cancer Res, 2003, 63 (14): p 4037-43). Our results demonstrate that there was a high level of PGE₂ production in SCC-25 cells and a significant reduction of PGE₂ synthesis in SCC-25 cells in the presence of Scutellaria baicalensis in a dose-dependent manner (FIG. 5).

We then examined the mechanism of inhibition of PGE₂ synthesis by Scutellaria baicalensis, i.e. direct inhibition of COX-2 enzymatic activity or inhibition of COX-2 expression. We measured COX-2 activity by in cell arachidonic acid conversion assay by providing exogenous arachidonic acid to the cells (Chen, Y., et al., Biochem Pharmacol, 2001. 61: p. 1417-1427). Our results showed that there was a significant decrease of PGE₂ level in the presence of both Scutellaria baicalensis and celecoxib, a COX-2 specific inhibitor (FIG. 6) (Zhang, D., et al., Cancer Res, 2003, 63 (14): p 4037-43). The pattern of PGE₂ inhibition was different between celecoxib and Scutellaria baicalensis: a significant delay in inhibition of PGE₂ synthesis by Scutellaria baicalensis indicated that it may have inhibited COX-2 expression directly. Therefore, we further examined the effect of Scutellaria baicalensis on COX-2 expression by Western blot. Our results showed that Scutellaria baicalensis inhibited protein expression (FIG. 7). These results demonstrated that Scutellaria baicalensis significantly inhibit COX-2 activity as well as COX-2 expression.

### Example 2.2

### Inhibition of PGE₂ Synthesis in Nude Mice

We also examined the effect of Scutellaria baicalensis on the PGE₂ production in animals bearing KB cells. The blood was collected at the end of experiment (8 weeks) and 50-ul plasma aliquots were subjected to PGE₂ analysis. Our results show that there is a significant decrease of plasma PGE₂ level in a dose-dependent manner, indicating that Scutellaria baicalensis can inhibit PGE₂ synthesis in animals (FIG. 8).

### Example 3

### Quality Control of Scutellaria baicalensis

### Example 3.1

### Chemical Analysis of Scutellaria baicalensis

The authenticity and chemical quality of Scutellaria baicalensis extract is determined by HPLC. HPLC analysis was performed using a Xterra-Rp C18 column (4.6 mm×250, 5 μm particle size) on a Perkin Elmer HPLC system, equipped with a chromatography interface 600 series link, a 888-PE pump, a 785A UV detector and an advanced LC sample processor ISS 200. 20 μl of each sample prepared by dissolving the herbal extracts in water or ethanol, and passed through filter was injected into the system. The peaks were detected at wavelength of 276 nm and assigned by comparison of the retention times and UV spectra.

We have tested Scutellaria baicalensis extracts purchased from five different companies (YF, STT, MW, MT and SC) in US. On HPLC profile, all extracts contained marker components, either baicalin or baicalein or both, confirming the extract's authenticity (FIG. 9). In addition, various amounts of other components were also observed in the extracts, further confirming the authenticity of these extracts. For example, oroxylin A 7-O-glcUA and wogonoside were found in extracts obtained from YF, STT and MW, while wogonin and oroxylin A were observed in extracts from MT and SC (FIG. 10). However, significant product-to-product and batch-to-batch variation regarding marker components was observed. Based on the presence of baicalin, the extracts can be separated into two groups: those with high baicalin concentrations (YF, STT, and MW) and those with minimal baicalin concentrations (MT and SC) (FIG. 10 and Table 2). Variation of baicalin concentrations was likely due to the solvent used in extract preparation. We found baicalin is the main constituent when the raw plant is extracted with water, while baicalein is the major constituent when extracted with ethanol.

Our HPLC study showed that although some variation in the amount of these marker components existed in different extracts, the presence of these marker components could be used to assess authenticity and chemical consistency of Scutellaria baicalensis extracts.

### Example 3.2

### Biological Activity Analysis of Scutellaria baicalensis

The variation of marker components in these extracts raised a significant concern about the bioactivity and potency of the herbal products. We have previously shown Scutellaria baicalensis inhibited COX-2 activity, thereby reducing PGE₂ synthesis (David Zhang et al. Cancer Res, 2003, 63 (14): 4037-43). Therefore, we tested the difference in their biological activity using PGE₂ synthesis by enzyme immunoassay (EIA). Our results showed all Scutellaria baicalensis extracts inhibited PGE₂ production in SCC-25 cells. However, significant variation (ranging from 62.3% to 94%) in the ability of inhibition of PGE₂ synthesis was observed among different products and batches (Table 2). Furthermore, no apparent relationship between the presence and amount of baicalin, baicalein or other components and the degree of PGE₂ inhibition was observed. These results indicated that baicalin, baicalein, or other components found by HPLC are not the components causing PGE₂ synthesis inhibition. These results indicate that measurement of marker components by HPLC does not reflect its biological activity, suggesting that while chemical standardization can provide some level of quality control, particularly authenticity, it is insufficient to determine biological activity due to complex chemical composition of an extract.

TABLE 2
Contents of baicalin and baicalein of ten commercial
extracts of Scutellaria baicalensis and inhibition of PGE₂
production in SCC-25 cells.

| | Baicalin | Baicalein | PGE₂ | |
|---|---|---|---|---|
| Sample | (μg/mg) | (μg/mg) | pg/10⁶ cells | reduction (%) |
| YF1 | 192.8 | 12.9 | 19.5 | 93.4 |
| YF2 | 143.8 | 11.7 | 34 | 88.4 |
| YF3 | 229.2 | — | 84.2 | 71.4 |
| STT1 | 148.8 | 12.0 | 96.3 | 67.3 |
| STT2 | 210.4 | 13.2 | 17.7 | 94.0 |
| MW1 | 120.0 | — | 111.1 | 62.3 |
| MT1 | — | 17.9 | 47.9 | 83.7 |
| MT2 | — | 33.9 | 23.9 | 91.9 |
| MT3 | — | 52.3 | 27.3 | 90.7 |
| SC1 | — | 33.0 | 37.4 | 87.3 |

### Example 4

### The Anti-Arthritic Activity of Scutellaria baicalensis Formula in Rat with Adjuvantive Arthritis

### Example 4.1

### Inhibition of Paw Edema Induced by Adjuvant

The anti-arthritic activity of Scutellaria baicalensis formula was confirmed in animal study. Eight week-old male Wistar rats were received intradermal injections of 0.1 ml Freund's complete adjuvant (FCA, 7.5 mg/ml) to the paw of the right hindfood. At 14 days, paw edema was induced and rats were randomized into three groups (n=6) including control group, Scutellaria baicalensis formula group (30 mg/kg of Scutellaria baicalensis, 10 mg/kg of Glucosamine and 8 mg/kg of Chondroitin) and indomethacin group (12.5 mg/kg) which serves as a positive control. The treatment groups were given Scutellaria baicalensis formula or indomethacin by oral gavage once per day for a duration of 10 days. The diameters of paw were measured at 16, 20 and 24 days. Our results showed that the diameters of paw significantly decreased in Scutellaria baicalensis formula group as compared with control group, and were similar to indomethacin group (Table 3). The result indicates that Scutellaria baicalensis formula has a strong anti-inflammatory activity in rats induced by adjuvant.

TABLE 3
The changes of paw edema after treating with Scutellaria baicalensis
formula and Indomethacin in rats.

| Groups | 16 d | 20 d | 24 d |
|---|---|---|---|
| control | 1.567 ± 0.121 | 1.883 ± 0.496 | 1.483 ± 0.366 |
| Arthrigia | 0.900 ± 0.141** | 0.817 ± 0.147** | 0.317 ± 0.172** |
| Indomethacin | 0.929 ± 0.214** | 0.771 ± 0.160** | 0.400 ± 0.245** |

X ± SD, n = 6,
**p < 0.01 compared with control group.

### Example 4.2

### The Effect of Scutellaria baicalensis Formula on IL-6 and PGE₂ in Rats with Adjuvant Arthritis

Osteoarthritis is characterized by articular cartilage degradation and hypertrophic joint changes. Pro-inflammatory cytokines (IL-1, IL-6, TNF-α) are considered to play important roles in the initiation and development of osteoarthritis (Inoue et al. British Journal of Pharmacology 136:287-295 (2002)). Prostaglandin (PG) E₂ has catabolic effects on the articular cartilage in OA (Futani et al J Immunother 2002 1;25(0):S61-S64). Therefore, we measured IL-6 and PGE₂ in rats' blood with adjuvant-induced arthritis treated with Scutellaria baicalensis formula using ELISA assay. At the end of the experiment in Example 4.1, IL-6 in serum and PGE2 in articular fluid were assessed. Our results showed that Scutellaria baicalensis formula significantly decreased IL-6 and PGE2, and was similar to that of indomethacin (Table 4).

TABLE 4
The effects of arthrigia on IL-6 and PGE2
in rats with adjuvantive arthritis

| Groups | IL-6 (pg/ml) | PGE2 (pg/ml) |
|---|---|---|
| Control | 17.9 ± 4.4 | 15.4 ± 6.8 |
| Arthrigia | 10.6 ± 3.6* | 8.7 ± 5.5* |
| Indomethacin | 6.0 ± 3.1** | 6.00 ± 3.1* |

X ± SD, n = 6,
*p < 0.05 or
**p < 0.01 compared with control group.

### Example 5

### Low Toxicity of Scutellaria baicalensis

Scutellaria baicalensis formula is composed of Glucosamine sulfate, Chondroitin sulfate and Scutellaria baicalensis. Many studies showed that no toxicity and side-effects for all these agents. No mortality was observed in mice or rats received glucosamine sulfate at very high levels (500 mg/kg oral, 3000 mg/kg IM, and 1500 mk/kg IV). Chondroitin sulfate is well tolerated following an oral dose and no signs or symptoms of toxicity have been reported (Conte A et al. Arzneim Forsch, 1995, 45: p 918-925). Our recent short and long term toxicity studies, evaluating hematology (WBC, RBC, and hemoglobin) (Table 5) and blood chemistry (creatine, BUN, AST, and ALT) in addition to body weight (FIG. 10) and vital organs (heart, liver, lung, kidney, and spleen) (FIG. 11), demonstrated no adverse effects after 8-week or 6-month treatment of Scutellaria baicalensis by oral gavage at dose of 1 g/kg (8.3-fold of human equivalent dose).

TABLE 5
The CBC and serum levels of nude mice treated
with Scutellaria baicalensis for 6 months

| | WBC | RBC | hemo- | | | | |
|---|---|---|---|---|---|---|---|
| | (×10³/ | (×10⁶/ | globin | Creatine | BUN | AST | ALT |
| | ul) | ul) | (g/dL) | (mg/dL) | (mg/dL) | (U/L) | (U/L) |
| control | 5.80 | 9.08 | 14.30 | 0.30 | 19.30 | 442.70 | 67.00 |
| 1000 | 6.60 | 8.92 | 14.30 | 0.30 | 20.30 | 327.30 | 54.00 |
| mg/kg | | | | | | | |
| SB | | | | | | | |

### Example 6

### Chemoprevention of Cigarette Smoke-Induced Oral Squamous Changes

### Example 6.1

### Inhibition of PaB Induced COX-2 Activity by Scutellaria baicalensis

Benzo[a]pyrene (BaP), one of the compounds released from smoking, is a prototypical carcinogen. It is a polycyclic aromatic hydrocarbon (PAH) compound and is found in high quantity (20-40 ng) in each cigarette. BaP is metabolized and activated to become the ultimate carcinogenic metabolite [(+)-anti-7b,8a-dihydroxy-9a,10a-epoxy-7,8,9,10-tetrahydorbenzo[a]pyrene], also known as BPDE. A recent study showed that BaP stimulates COX expression both in normal and transformed oral squamous epithelial cells (Kelley, D et al. Carcinogenesis, 1997. 18(4): p. 795-799). We examined the counter-stimulatory effect of Scutellaria baicalensis on BaP-induced COX-2 activity. After the treating SCC-25 cells with BaP for 6 hours, the supernatants were removed and the cells were washed with PBS. Fresh medium containing 0, 75, 150, 300 ug/ml SB or 25 uM celecoxib (previously determined IC₅₀) was added and incubated for additional 24 hours [1]. This situation resembles the chemoprevention in the former smokers. Our results show that both Scutellaria baicalensis and celecoxib inhibit PGE₂ synthesis induced by BaP and the inhibition is so strong that PGE₂ level drop below the baseline to an undetectable level, suggesting both agents also suppress endogenous COX-2 activity (FIG. 12). Furthermore, the ability for celecoxib to inhibit PGE₂ synthesis supports our hypothesis that COX-2 is the enzyme responsible for the increase of PGE₂ induced by BaP. These results further support our rationale of using Scutellaria baicalensis as a chemoprevention agent.

### Example 6.2

### Inhibition of BaP-Induced Squamous Epithelial Changes by Scutellaria baicalensis

Two nude mice were treated by apical application of BaP (0.5 ug/ml) to the buccal mucosa with a cotton swab daily for 6 days. The animals were sacrificed at the end of the experiment and the buccal tissue was removed and embedded in paraffin. Sections from control and treated animals were stained with H&E and examined under a light microscope. The results demonstrated hyperkeratosis, an early change of BaP-induced tissue damage, of the buccal mucosa in the treated animals (FIG. 13). This study confirmed that BaP cause cellular and histological changes in a murine animal model.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A composition formulated by extracting a therapeutically effective amount of Scutellaria baicalensis.

15. The composition of claim 14, wherein the composition is capable of treating cancer.

16. The composition of claim 14, wherein the composition is capable of preventing cancer.

17. The composition of claim 14, further comprising a therapeutically effective amount of glucosamine and chondroitin.

18. The composition of claim 17, wherein the composition comprises 20 to 40% Scutellaria baicalensis.

19. The composition of claim 17, wherein the composition comprises about 34% Scutellaria baicalensis.

20. The composition of claim 17, wherein the composition comprises 30 to 60% glucosamine.

21. The composition of claim 17, wherein the composition comprises about 52% glucosamine.

21. The composition of claim 17, wherein the composition comprises 10 to 20% chondroitin.

22. The composition of claim 17, wherein the composition comprises about 14% chondroitin.

23. The composition of claim 17, wherein the composition is capable of treating arthritis.

24. The composition of claim 17, wherein the composition is capable of preventing arthritis.

25. The composition of claim 23, wherein the arthritis comprises rheumatoid arthritis and osteoarthritis.

26. A method of treating a subject comprising administering an effective amount of the composition of claim 1.

27. A method of treating a subject comprising administering an effective amount of the composition of claim 4.

28. A method for determining Scutellaria baicalensis batch variation, the method comprising:
determining physical characteristics of the Scutellaria baicalensis batch; and
determining biological activity of the Scutellaria baicalensis batch.

29. The method of claim 28, wherein determining the physical characteristics comprises using high pressure chromatography.

30. A method for forming the composition of claim 1 comprising performing an extraction of Scutellaria baicalensis.

31. The method of claim 30, further comprising combining the Scutellaria baicalensis extract with glucosamine and chondroitin.
